(19) United States
(12) Patent Application Publication
(10) Pub. No.: US 2009/0004290 A1
(43) Pub. Date: Jan. 1, 2009

(54) RED YEAST RICE COMPOUND FOR CANCER CHEMOPREVENTION

(76) Inventor: Kirk G. Voelker, Sarasota, FL (US)

Correspondence Address: KIRK G. VOELKER, 1537 STATE ST., SARAGOTA, FL, 34236, US

(21) Appl. No.: 11/772,122

(22) Filed: Jun. 30, 2007

(51) Int. Cl. A61K 36/06; A61K 31/355; A61K 31/5025; A61K 31/60; A61K 31/714; A61P 35/00; A61K 36/82; A61K 33/04; A61K 33/30; A61K 33/34

(52) U.S. Cl. 424/630; 424/195.16; 424/641; 424/702; 424/729; 514/165; 514/249; 514/458; 514/52

(57) ABSTRACT

The invention concerns a composition containing red yeast rice and other components, as a chemopreventative agent to reduce the risk of cancer.

### FIELD OF THE INVENTION

Lung cancer is the number one cause of cancer death. Lung cancer accounts for more cancer deaths than breast, colon, prostate and ovarian cancer combined. Chemoprevention, the use of agents to prevent, reduce or reverse carcinogenesis, has been a focus of research for decades. Many agents that have been explored to date have been ineffective or marginally effective. Some studies have even demonstrated a possible increase in cancer risk with chemopreventative agents such as beta carotine.

### BACKGROUND OF THE INVENTION

Recent research has demonstrated that 3-Hydroxy-3-methylglutaryl coenzyme A reductase inhibitors or “statin” drugs may have antitumor effects. They have been shown to increase apoptosis and decrease angiogenesis through their effects on vascular endothelial growth factor. There is also evidence that statin drugs may alter tumor invasion and metastasis through their interaction with adhesion molecules.

Until recently, clinical trials have not demonstrated a significant beneficial effect using statin drugs as chemopreventative. A metaanalysis of 35 randomized controlled studies has found nor benefit or detriment using statins as a chemopreventative agent. In May 2007 Khurana et al published a retrospective case controlled study of >400,000 patients in the Veteran's Administration Database determining the potential impact of statins on lung cancer patients. This study found a 55% reduction in lung cancer risk in patients taking statins for >6 months.

Red yeast rice (RYR) is the product of yeast (Monascus purpureus) grown on rice, and is a dietary staple in some Asian countries. Red yeast rice has been used in China for over 1,000 years for medicinal purposes, and continues to be a dietary staple in China, Japan, and Asian communities in the United States. In these communities, the estimated average consumption of 14 to 55 grams of red yeast rice per day per person.

Processed red yeast rice supplements include red yeast rice extract (RYRE), which is any extract of red yeast rice, and xuezhikang, an alcohol extract of red yeast rice. Red yeast rice contains several compounds collectively known as monacolins, substances known to inhibit cholesterol synthesis. One of these, monacolin K, has the same chemical structure as the drugs lovastatin and mevinolin, potent inhibitors of HMG-CoA reductase and therefore can be considered a naturally occurring statin.

Selenium has been studied as a chemopreventative agent for over thirty years. Recently the Nutritional Prevention of Cancer Trial (NPC Trial) has demonstrated a significant decrease in lung cancer with selenium supplementation. Based upon the follow up of over 1300 participants, a secondary endpoint of the NPC Trial demonstrated a hazard ratio of 0.56 in those who received selenium supplementation. After reanalysis of these patients at almost 8 years the relative risk reduction of 0.70 was not statistically significant. However, subset analysis demonstrated that selenium appeared to have a chemopreventative effect on heavy smokers with low selenium levels (below 106 ng/ml).

Selenium, Zinc and Copper are all cofactors for several important enzymes involved in maintaining DNA integrity. An interim analysis of over 3200 participants in an ongoing study of selenium, zinc and copper supplementation, demonstrated that selenium supplementation is associated with a significant inverse trend (p<0.04) of lung cancer in men. There was also a significant chemoprotective trend (p<0.05) against lung cancer with increasing dietary zinc. Increased copper intake was also associated with a protective trend (p<0.05) against lung cancer.

Alpha-tocopherol (Vitamin E) has been implicated as a chemopreventative agent. In the only published randomized control trial there was a higher mortality due to hemorrhagic stroke among the participants that received Alpha-tocopherol. However, in the same study there was a 19% decrease incidence of lung cancer in participants with the highest blood levels of Alpha-tocopherol when compared to the quartile with the lowest levels. Alpha-tocopherol was found to be more protective in younger men with a fewer pack year history of smoking suggesting that Alpha-tocopherol may be beneficial in inhibiting lung cancer in the earlier stages.

In a double blinded study by Heimburger et al, Folate and Vitamin B12 supplementation has been shown to reverse bronchial epithelial cellular atypia and metaplasia in smokers.

An empirical link between green tea and its cancer prevention properties was made in the late 1980s. Epidemiological studies show that cancer onset of patients in Japan who had consumed ten cups of green tea per day was 8.7 years later among females and 3 years later among males, compared with patients who had consumed under three cups per day. Epigallocatechin gallate (EGCg), the major catechin in green tea, has been the focus of many studies. The administration of a pharmacologically effective amount of EGCg has been alleged to reduce the incidence of lung cancer in a mammal. A bioavailability study showed that frequent green tea consumption results in high levels of EGCg in various body organs, suggesting that green tea consumption may protect against cancers localized to different sites of the body. Green tea polyphenols have demonstrated a significant effect on tumor TGF beta expression and cell cycle regulatory proteins, giving it potential for chemoprevention targeted at the cell cycle regulatory pathway genes in cancer.

The relationship between nonsteroidal anti inflammatory drugs (NSAIDs), including aspirin, and cancer has been studied in several epidemiological studies with promising results to indicate that this class of drug may be beneficial in cancer chemoprevention.21 One case-control study subgroup of the New York University Women's Health Study, suggest that regular aspirin use might be inversely associated with risk of lung cancer in women, particularly the non-small cell sub-type In the Women's Health Study, sponsored by the National Institutes of Health, 40,000 apparently healthy women health professionals ages 45 and older were being assigned at random to 50 mg beta carotene, 600 IU alpha tocopherol, 100 mg aspirin, and/or placebos every other day. The beta carotene portion of this trial was dropped. Results from this large-scale, long-term trial suggest that alternate day use of low-dose aspirin (100 mg) for an average 10 years of treatment does not lower risk of total, breast, colorectal, or other site-specific cancers. The study concluded “A protective effect on lung cancer or a benefit of higher doses of aspirin cannot be ruled out.”

Though the above information is focused primarily at lung cancer, this does not preclude the benefit of the composition in other forms of chemoprevention including, but are not limited to, carcinoma and sarcoma such as leukemia, sarcoma, osteosarcoma, lymphomas, melanoma, glioma, pheochromocytoma, hepatoma, ovarian cancer, skin cancer, testicular cancer, gastric cancer, pancreatic cancer, renal cancer, breast cancer, prostate cancer, colorectal cancer, cancer of head and neck, brain cancer, esophageal cancer, bladder cancer, adrenal cortical cancer, endometrial cancer, nasopharyngeal cancer, cervical or liver cancer, and cancer of unknown primary site.

### Definitions

Unless the context specifies otherwise, the word “comprise” or variations of such, or the word “includes” or variations of such, or the term “having” or variations of such, will be understood to imply the inclusion of a stated element or integer or group of elements or integers but not the exclusion of any other element or integer or group of elements or integers.

The terms “red yeast rice,” “red yeast rice product”, “red yeast rice extract” and the like refer to a product that results from the fermentation of at least one Monascus. Further, these latter terms include traditional and improved red rice products as described below. More specifically, “red rice product” as used herein refers to the product of fermentation, e.g., the fermentate of one or a mixture of Monascus fungus.

Vitamin B12 encompasses cyanocobalamin in all physiologically acceptable forms (such as hydroxocobalamin) which may be used in the formulation of the present invention.

Aspirin includes, but is not limited to, all forms of acetylsalicylic acid including buffered aspirin, enteric coated aspirin, aspirin salts such as calcium acetylsalicylate, and mixtures of aspirin with acid acceptors any of which may be used in the formulation of the present invention.

The term green tea or green tea extract, encompasses the polyphenols in green tea that have been identified including Polyphenon E, catechin (C), epicatechin (EC), gallocatechin (GC), gallocatechin gallate (GCG), epigallocatechin (EGC), epicatechin gallate (ECG), and epigallocatechin gallate (EGCg). In addition, caffeine, theobromine, theophylline, and phenolic acids, such as gallic acid, may also be present as constituents of green tea in smaller quantities than the polyphenols.

Unless the context specifies otherwise, the word “composition” or variations of such will be understood to imply the formentioned elements or integers, groups of elements or integers may be administered individually or compounded together. They may be temporally administered together or sequentially.

The term “chemoprevention” or variations of such will be understood to imply the use of agents to prevent, reduce or reverse carcinogenesis or metastasis of cancer.

### SUMMARY OF THE INVENTION

In this invention, ingredients were specifically chosen based on the weight of scientific evidence in existing clinical trials and peer-reviewed research, particularly in clinical trials of cancer prevention. The individual ingredients were also selected based on their theoretical mechanism of action, to be synergistic in chemoprevention. This synergy has the potential to improve the efficacy of a component that has only marginal chemopreventive properties. Although each ingredient selected for the preferred embodiment has been used before for possible cancer prevention, cancer risk reduction or their effect on regulating the promotion of carcinogenesis, their total combination for cancer prevention is a new concept. Though the research presented is by no means all encompassing but is rather focused on the chemopreventative effects in lung cancer, the scope of this invention is by no means limited to malignancies of the respiratory system.

### DETAILED DESCRIPTION

The composition is administered orally for individuals who wish to reduce their risk of disease, particularly cancer-risk.

Broadly, compositions of this invention are edible, that is to say, they are suitably formulated for oral use (e.g., chewing gum, tablets, lozenges, capsules, elixirs and the like). Regardless of the particular form, unless otherwise indicated overtly or by omission, the compositions consist essentially of a base of red yeast rice, a product of the fermentation of at least one Monascus stain plus an additional that can be used as a dietary supplement or as a therapeutic medicament.

The invention is based upon a formulation which may comprise, consist essentially of Red yeast rice, selenium, Alpha tocopherol, zinc, copper, folic acid, B12, aspirin, green tea extract or any combination thereof. Advantageously, dosage per caplet or tablet of red yeast rice is 600 mg, selenium is 50 mg, Alpha tocopherol 20 mg, vitamin B12 is 200 mcg, folic acid or folate is 2 mg, tea phenol is 200 mg, zinc 3 mg copper 2 mg. The caplet or tablet of may further comprise cellulose, silica and magnesium stearate. In another embodiment, the caplet or tablet may further comprise calcium carbonate, carnauba wax, colloidal silicon dioxide, crospovidone, hypromellulose, lactose, magnesium stearate, maltodextrin, microcrystalline cellulose, pregelatinized starch, sodium starch glycolate, stearic acid, titanium dioxide, tracetin, zinc stearate or any combination thereof. Preferably, the caplet or tablet does not contain gluten, preservatives, sugar, sodium, milk, yeast, artificial colors, artificial flavors or any combination thereof.

In a further embodiment, the composition is administered in four tablets, each comprising about 600 mg red yeast rice, about 50 mcg selenium to provide a total daily dose of about 2.4 gm red yeast rice, about 200 mcg selenium

In a further embodiment, the composition is administered in four tablets, each comprising about 600 mg red yeast rice, about 50 mcg selenium, and about 20 mg alpha tocopherol to provide a total daily dose of about 2.4 gm red yeast rice, about 200 mcg selenium and about 80 mg alpha tocopherol

In a further embodiment, the composition is administered in four tablets, each comprising about 600 mg red yeast rice, about 50 mcg selenium, vitamin B12 is 200 mcg, folic acid or folate is 2.5 mg to provide a total daily dose of about 2.4 gm red yeast rice, about 200 mcg selenium, vitamin B12 is 800 mcg, folic acid or folate is 10 mg

In a further embodiment, the composition is administered in four tablets, each comprising about 600 mg red yeast rice, about 50 mcg selenium, about 3 mg zinc and about 0.3 mg copper to provide a total daily dose of about 2.4 gm red yeast rice, about 200 mcg selenium, about 12 mg zinc and about 1.2 mg copper.

In a further embodiment, the composition is administered in four tablets, each comprising about 600 mg red yeast rice, about 50 mcg selenium, vitamin B12 is 200 mcg, folic acid or folate is 2.5 mg, zinc is about 3 mg and copper is about 0.3 mg to provide a total daily dose of about 2.4 gm red yeast rice, about 200 mcg selenium, vitamin B12 is 800 mcg, folic acid or folate is 10 mg, about 12 mg zinc and about 1.2 mg copper per day.

In a further embodiment, for example, for persons already ingesting a 3-Hydroxy-3-methylglutaryl coenzyme A reductase inhibitor for other reasons, the composition is administered in four tablets, each comprising about 50 mcg selenium, vitamin B12 is 200 mcg, folic acid or folate is 2.5 mg zinc is about 3 mg and copper is about 0.3 mg to provide a total daily dose of about 200 mcg selenium, vitamin B12 is 800 mcg, folic acid or folate is 10 mg, about 12 mg zinc and about 1.2 mg copper per day.

In other embodiments, aspirin and green tea extract are added for their additional chemoprevention properties.

Tablets contain the active ingredients in admixture with nontoxic pharmaceutically acceptable excipients which are suitable for manufacture of tablets. These excipients may be, inert diluents, for example, calcium carbonate, sodium carbonate, lactose, calcium phosphate or sodium phosphate; granulating and disintegrating agents, for example, alginic acid, croscarmellose sodium, maize starch or; binding agents, for example, acacia, gelatine or starch, and lubricating agents, for example, magnesium stearate or stearic acid. The tablets may be uncoated or they may be coated by known techniques to delay disintegration and absorption in the gastrointestinal tract and thereby provide an even longer sustained action over a period of time. The tablets may be chewable or non-chewable and designed to desired weight, potency and hardness through well known skills in the pharmaceutical arts.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredient is mixed with a suitable oil medium, for example, arachis oil, liquid paraffin or olive oil.

Formulations for oral use may also be presented as lozenges wherein the active ingredients are mixed into a hard candy composition. Suitable hard candy compositions can be made from varying, but highly concentrated, sucrose solutions including corn syrup as a second essential ingredient. Other known hard candy compositions may utilize any suitable good testing, sweet excipient other than sucrose.

Moreover, where in tablet or pill form the compositions can be coated to delay disintegration and absorption in the gastrointestinal tract thereby providing a sustained action over an extended period of time. Selectively permeable membranes surrounding an osmotically active driving compound are also suitable for orally administered compositions. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A nine delay material such as glycerol monostearate or glycerol stearate may also be used.

In yet another less preferred embodiment, the compounds of the invention can be delivered in a controlled release system. In another embodiment, polymeric materials can be used. In yet another embodiment, a controlled-release system can be placed in the individual compounds of the composition may be administered either substantially together or simultaneously in separate or combined formulations.

By “substantially together”, the active ingredients of the composition of the invention are administered to a person in separate dosage forms, such that, the active ingredients are administered either simultaneously or within a period of time such that the person receives benefit of the aggregate effects of the separate dosage forms. For example, the active ingredients may be taken together or within a few seconds to at least about 60 minutes of one another. Accordingly, methods of treatment of the present invention, therefore, include administration of the individual compounds of such combinations either substantially together or simultaneously in separate or combined pharmaceutical formulations. When administering or taking the active ingredients substantially together, but separately in same or different dosage forms, the order in which they are administered or ingested is not critical.

The composition may also be administered temporally apart. By “temporally apart”, the active ingredients of the composition of the invention are administered to a person in separate dosage forms at different times, such that, the active ingredients are administered outside of a period of time such that the aggregate effects of the separate dosage forms is not dependent on administration of the components “substantially together” but rather on steady state pharmacodynamics. For example, the active ingredients may be taken as an “AM” and “PM” dosage.

Aqueous suspensions contain the active ingredients in admixture with excipients suitable for the manufacture of aqueous suspensions. The aqueous suspensions may also contain one or more suitable preservatives, for example, ethyl, or n-propyl, p-hydroxy benzoate, one or more suitable coloring agents, one or more suitable flavoring agents such as, cinnamon, chocolate, fruit flavors (i.e., cherry, grape, orange, strawberry, etc.), menthol, mints, vanilla and combination of two or more thereof, one or more suitable sweetening agents, such as calcium cyclamate, dextrose, fructose, galactose, glucose, glycerin, maltose, mannitol, mannose, ribose, partially hydrolyzed starch solids, partially hydrolyzed corn syrup solids, sodium cyclamate, sorbital, inulin, sucralose, sucrose, xylitol, or xylose, and one or more suitable coloring agents.

What is claimed is:

1. A method preventing or impeding the growth of a neoplastic condition in a human comprising the administration to said human a composition comprising between 10 mg and 200 gm per day of red yeast rice, and at least one additional chemopreventitive agent selected from the group consisting of selenium, alpha tocopherol, Vitamin B12, folate, copper, zinc, green tea extract, and aspirin, wherein said composition is in a delivery device selected from the group consisting essentially of a tablet, a capsule, a solution, a suspension, an emulsion, a foodstuff, a pharmaceutical composition, a dietary supplement composition, and a nutritional supplement composition.

2. A composition according to claim 1 in which the additional chemopreventative agent is selenium in an amount between 50 mcg and 1000 mcg per day.

3. A composition according to claim 1 in which the additional chemopreventative agents comprise selenium in an amount between 50 mcg and 1000 mcg per day and alpha tocopherol in an amount between 50 IU and 5000 IU per day.

4. A composition according to claim 1 in which the additional chemopreventative agents comprise selenium in an amount between 50 mcg and 1000 mcg per day, alpha tocopherol in an amount between 50 IU and 5000 IU per day, Vitamin B12 in an amount between 50 and 5000 micrograms per day and folate in an amount between 1 and 100 mg per day.

5. A composition according to claim 1 in which the additional chemopreventative agents comprise selenium in an amount between 50 mcg and 1000 mcg per day, alpha tocopherol in an amount between 50 IU and 5000 IU per day, Vitamin B12 in an amount between 50 and 5000 micrograms per day, folate in an amount between 1 and 100 mg per day, copper in an amount between 0.2 mg and 20 mg per day and zinc in an amount between 0.4 mg and 40 mg per day.

6. A composition according to claim 1 in which the additional chemopreventative agents comprise selenium in an amount between 50 mcg and 1000 mcg per day, alpha tocopherol in an amount between 50 IU and 5000 IU per day, Vitamin B12 in an amount between 50 and 5000 micrograms per day, folate in an amount between 1 and 100 mg per day, copper in an amount between 0.2 mg, 20 mg per day and zinc in an amount between 0.4 mg and 40 mg per day and green tea extract in an amount between 40 mg and 4000 mg per day.

7. A composition according to claim 1 in which the additional chemopreventative agents comprise selenium in an amount between 50 mcg and 1000 mcg per day, alpha tocopherol in an amount between 50 IU and 5000 IU per day, Vitamin B12 in an amount between 50 and 5000 micrograms per day, folate in an amount between 1 and 100 mg per day, copper in an amount between 0.2 mg, 20 mg per day and zinc in an amount between 0.4 mg and 40 mg per day and green tea extract in an amount between 40 mg and 4000 mg per day and aspirin in an amount between 10 and 1000 mg per day.

8. A composition according to claim 1 in which the additional chemopreventative agents comprise selenium in an amount between 50 mcg and 1000 mcg per day, Vitamin B12 in an amount between 50 and 5000 micrograms per day and folate in an amount between 1 and 100 mg per day.

9. A composition according to claim 1 in which the additional chemopreventative agents comprise Vitamin B12 in an amount between 50 and 5000 micrograms per day, folate in an amount between 1 and 100 mg per day, copper in an amount between 0.2 mg and 20 mg per day and zinc in an amount between 0.4 mg and 40 mg per day.

10. A composition according to claim 1 in which the additional chemopreventative agents comprise Vitamin B12 in an amount between 50 and 5000 micrograms per day, folate in an amount between 1 and 100 mg per day, copper in an amount between 0.2 mg, 20 mg per day and zinc in an amount between 0.4 mg and 40 mg per day and green tea extract in an amount between 40 mg and 4000 mg per day.

11. A composition according to claim 1 in which the additional chemopreventative agents comprise green tea extract in an amount between 40 mg and 4000 mg per day.

12. A composition according to claim 1 in which the additional chemopreventative agents comprise selenium in an amount between 50 mcg and 1000 mcg per day and green tea extract in an amount between 40 mg and 4000 mg per day.

13. A composition according to claim 1 in which the additional chemopreventative agents comprise selenium in an amount between 50 mcg and 1000 mcg per day, Vitamin B12 in an amount between 50 and 5000 micrograms per day, folate in an amount between 1 and 100 mg per day, copper in an amount between 0.2 mg, 20 mg per day and zinc in an amount between 0.4 mg and 40 mg per day and green tea extract in an amount between 40 mg and 4000 mg per day.

14. A composition according to claim 1 in which the additional chemopreventative agents comprise Vitamin B12 in an amount between 50 and 5000 micrograms per day and folate in an amount between 1 and 100 mg per day.

15. A composition according to claim 1 in which the additional chemopreventative agents comprise between trace amounts and 1000 mcg per day of selenium, between trace amounts and 5000 IU per day of alpha tocopherol, between trace amounts and 5000 micrograms of Vitamin B12 per day and between trace amounts and 100 mg folate per day, between trace amounts and 20 mg of copper per day, between trace amounts and 40 mg zinc per day, between trace amounts and 4000 mg of green tea extract per day, wherein said composition is in a delivery device selected from the group consisting essentially of a tablet, a capsule, a solution, a suspension, an emulsion, a foodstuff, a pharmaceutical composition, a dietary supplement composition, and a nutritional supplement composition.

16. A method preventing or impeding the growth of a neoplastic condition in a human comprising the administration to said human a composition comprising at least two chemopreventitive agent selected from the group consisting of selenium, alpha tocopherol, Vitamin B12, folate, copper, zinc, green tea extract, and aspirin, wherein said composition is in a delivery device selected from the group consisting essentially of a tablet, a capsule, a solution, a suspension, an emulsion, a foodstuff, a pharmaceutical composition, a dietary supplement composition, and a nutritional supplement composition.

17. A composition according to claim 16 in which the chemopreventative agents comprise between trace amounts and 1000 mcg per day of selenium, between trace amounts and 5000 IU per day of alpha tocopherol, trace amounts and 5000 micrograms of Vitamin B12 per day and between trace amounts and 100 mg folate per day, between trace amounts and 20 mg of copper per day, between trace amounts and 40 mg zinc per day, between trace amounts and 4000 mg of green tea extract per day.

18. A composition according to claim 16 in which the chemopreventative agents comprise between 50 mcg and 1000 mcg per day of selenium, between 50 IU and 5000 IU per day of alpha tocopherol, between 50 and 5000 micrograms of Vitamin B12 per day and between 1 and 100 mg folate per day, between 0.2 mg and 20 mg of copper per day, between 0.4 mg and 40 mg zinc per day and between 40 mg and 4000 mg of green tea extract per day.

19. A composition according to claim 16 in which the chemopreventative agents comprise between 50 mcg and 1000 mcg per day of selenium, between 50 IU and 5000 IU per day of alpha tocopherol, between 50 and 5000 micrograms of Vitamin B12 per day and between 1 and 100 mg folate per day, between 0.2 mg and 20 mg of copper per day and between 0.4 mg and 40 mg zinc per day.

20. A method preventing or impeding the growth of a neoplastic condition in a human comprising the administration to said human a composition comprising between 10 mg and 200 gm per day of red yeast rice, selenium in an amount between 50 mcg and 1000 mcg per day, Vitamin B12 in an amount between 50 and 5000 micrograms per day and folate in an amount between 1 and 100 mg per day, wherein said composition is in a delivery device selected from the group consisting essentially of a tablet, a capsule, a solution, a suspension, an emulsion, a foodstuff, a pharmaceutical composition, a dietary supplement composition, and a nutritional supplement composition.
